(19) United States
(12) Patent Application Publication
(10) Pub. No.: US 2020/0260989 A1
(43) Pub. Date: Aug. 20, 2020

(54) ENDOSCOPE LOCALIZATION USING TRANSMISSION ULTRASONOGRAPHY

(71) Applicant: THE JOHNS HOPKINS UNIVERSITY, Baltimore, MD (US)

(72) Inventors: James Paul Taylor, Baltimore, MD (US); Seth Daniel Goldstein, Baltimore, MD (US)

(21) Appl. No.: 16/644,911

(22) PCT Filed: Aug. 8, 2018
(86) PCT No.: PCT/US2018/045846

§ 371 Date: Mar. 5, 2020

Related U.S. Application Data
(60) Provisional application No. 62/555,834, filed on Sep. 8, 2017.

(51) Int. Cl. A61B 5/06; A61B 1/31; A61B 1/00; A61B 1/06; A61B 1/04; A61B 10/04; A61B 5/00

(52) U.S. Cl. CPC A61B 5/066; A61B 1/31; A61B 1/00137; A61B 5/6823; A61B 1/042; A61B 10/04; A61B 1/063

(57) ABSTRACT

A system for determining a location of a tool within a patient includes a tool and a sensor. The tool is configured to be introduced into a patient. The tool includes a flexible tubular member, a laser coupled to the flexible tubular member, and a cap coupled to the laser. A plurality of waves are generated when a beam of light from the laser shines upon the cap. The sensor is configured to be coupled to an exterior of the patient. The sensor is configured to receive the waves, and a location of the tool within the patient is configured to be determined based at least partially upon the waves received by the first sensor.

### CROSS-REFERNCE TO RELATED APPLICATIONS

This application claims priority to U.S. Provisional Patent Application No. 62/555,834, filed on Sep. 8, 2017, the entirety of which is incorporated by reference herein.

### FIELD OF THE INVENTION

The present invention relates generally to identifying the location of a tool within a patient. More particularly, the present invention relates to identifying the location of a colonoscope within a human colon.

### BACKGROUND OF THE INVENTION

A colonoscope is used to visualize a patient's colon. The colonoscope is introduced into the patient through the anus and “snakes” its way through the twists and turns of the colon. The colonoscope includes a camera at a distal end thereof. The camera captures images/video as the colonoscope moves throughout the colon. When the images/video show an abnormality that may be, for example, colorectal cancer, surgery may be performed to remove or otherwise address the abnormality. However, due to the many twists and turns of the colon, it may be difficult to pinpoint the exact location of the colonoscope and the abnormality. As a result, it may be difficult for the surgeon to know where to operate. This is particularly true during minimally-invasive surgeries where the surgeon has a limited view and lack of tactile sensation.

### SUMMARY OF THE INVENTION

A system for determining a location of a tool within a patient is disclosed. The system includes a tool and a sensor. The tool is configured to be introduced into a patient. The tool includes a flexible tubular member, a laser coupled to the flexible tubular member, and a cap coupled to the laser. A plurality of waves are generated when a beam of light from the laser shines upon the cap. The sensor is configured to be coupled to an exterior of the patient. The sensor is configured to receive the waves, and a location of the tool within the patient is configured to be determined based at least partially upon the waves received by the first sensor.

A method for operating a device within a patient is also disclosed. The method includes introducing a device into a patient. The device includes a first bore having a location tool positioned therein. A plurality of waves are generated with the location tool when the device is positioned within the patient. The waves are received with a sensor coupled to an exterior of the patient. A position of the device within the patient is determined based at least partially upon the waves received by the sensor.

### BRIEF DESCRIPTION OF THE DRAWINGS

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a perspective view of a portion of an intraluminal device that is configured to be used inside a hollow organ in a patient.

FIG. 2 illustrates a side view of a location tool that is positioned within a bore of the device of FIG. 1.

FIG. 3 illustrates a portion of an abdomen of a patient with one or more sensors coupled thereto.

FIG. 4 illustrates a screen showing data from the device and the sensors.

FIG. 5 illustrates a perspective view of the device from FIG. 1 with an endocuff coupled to an end of the device.

FIG. 6 illustrates a flowchart of a method for operating the device within a patient.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

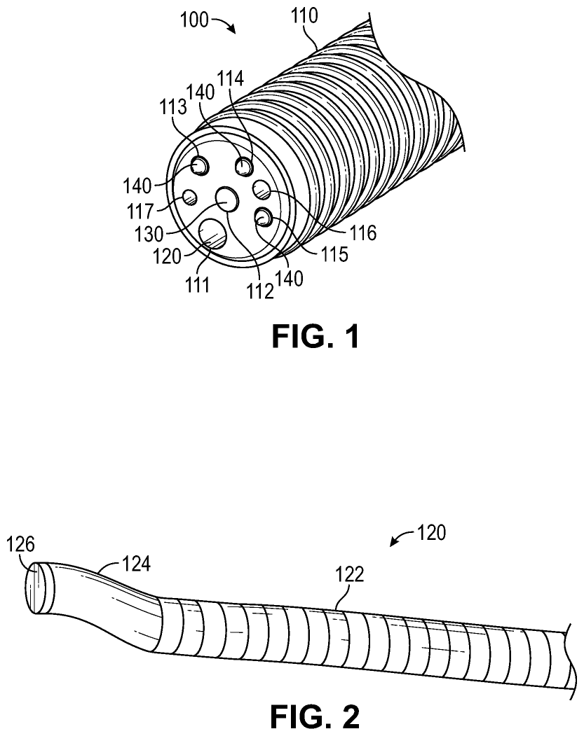

FIG. 1 illustrates a perspective view of a portion of an intraluminal device 100 that is configured to be used inside a hollow organ in a patient. The device 100 may be a colonoscope, sigmoidoscope, proctoscope, gastroscope, or other endoscope meant for intraluminal use in the patient's intestine. The device may be end-viewing or side-viewing with respect to the fiberoptic portion. There may be an endocuff or other after-market device installed on the endoscope, as described below.

The device 100 may be or include a flexible tubular member 110 having one or more bores (seven are shown: 111-117) formed axially-therethrough. The first bore 111 may be referred to as a working bore or working channel. The first bore 111 may be configured to have a location tool 120 positioned therein. The location tool 120 may be configured to transmit (e.g., ultrasound) waves that may be detected by one or more sensors coupled to an exterior of a patient to identify the precise location of the location tool 120 (and the device 100) within the interior of the patient, as described in greater detail below. In another example, the tool 120 may be withdrawn from the first bore 111 and a second tool (not shown) may be introduced into the first bore 111. The second tool may be or include a biopsy tool that is configured to remove a portion of tissue (e.g., a polyp) from inside the patient's rectum, colon, cecum, ileum, jejunum, duodenum, or stomach. The removed portion of tissue may be analyzed after the device 100 is withdrawn from the patient (e.g., to determine of the tissue is benign or malignant).

The second bore 112 may have a camera 130 positioned therein. The camera 130 may be or include a fiber optic camera or a charge-coupled device (CCD) camera. The camera 130 may be configured to capture images and/or video inside the patient (e.g., inside the patient's rectum, colon, etc.). The images and/or video may be used to provide a visual diagnosis (e.g., to identify polyps).

One or more of the bores (e.g., bores 113-115) may have lights 140 positioned therein. The lights 140 may be configured to illuminate the interior of the patient for the camera 130. One or more of the bores (e.g., bores 116, 117) may be configured to introduce air and/or water into the patient.

FIG. 2 illustrates a side view of the location tool 120. The location tool 120 may include a flexible tubular member 122 that is configured to bend or flex together with the device 100 when the location tool 120 is positioned within the first bore 111 of the device 100. The location tool 120 may also include a laser 124 that is coupled to a distal end of the tubular member 122. The laser 124 may be configured to emit a beam of light through a distal end thereof. However, a (e.g., metallic) cap 126 may be positioned over the distal end of the laser 124. The cap 126 may partially or completely cover the distal end of the laser 124 and thus partially or completely obstruct the beam of light. Thus, in one embodiment, the beam of light from the laser 124 may not shine on or otherwise illuminate the interior of the patient's body when the laser 124 is turned on.

One or more (e.g., ultrasound) waves may be generated in response to the beam of light from the laser 124 shining on the cap 126. The waves may have a wavelength from about 100 kHz to about 100 MHz.

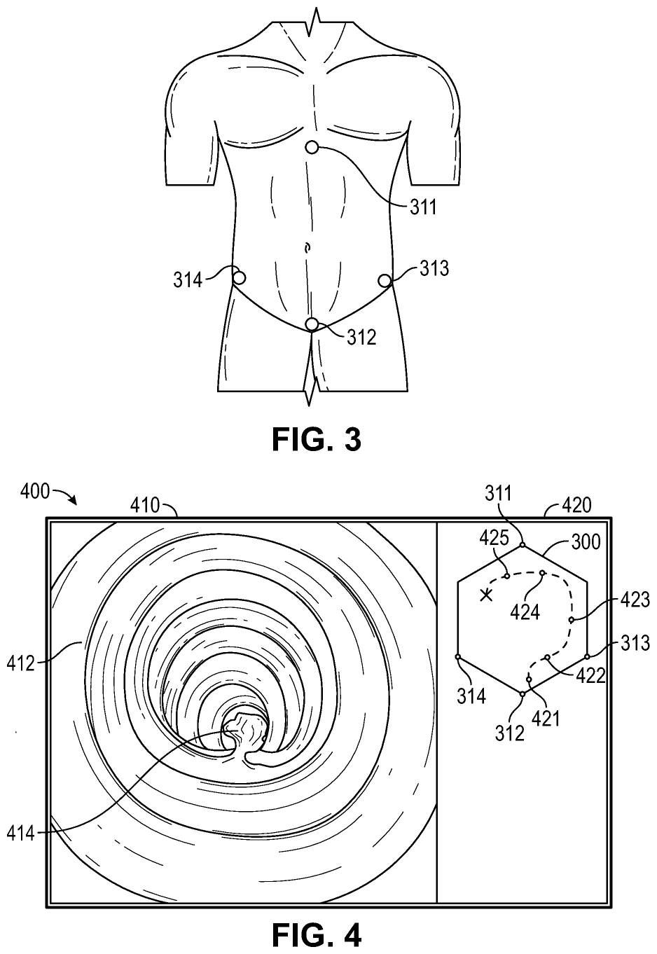

FIG. 3 illustrates a portion of a patient's abdomen 300 with one or more sensors (four are shown: 311-314) coupled thereto. The sensors 311-314 may be or include piezoelectric sensors that are coupled to the exterior of the abdomen 300. As shown, the first sensor 311 may be coupled to the sternum, the second sensor 312 may be coupled to the pubis, and the third sensor 313 may be coupled to one side of the iliac crest, and the fourth sensor 314 may be coupled to the other side of the iliac crest. The received waves enable a user (e.g., a doctor) to determine the precise location of the location tool 120 (and thus the device 100) within the abdomen 300 via photo-acoustic imaging. More particularly, as the waves all originate from the same source (e.g., the location tool 120), the phases of the waves may be compared at each sensor 311-314 to enable the user to triangulate the location of the location tool 120 (and thus the device 100) within the abdomen 300. As each sensor 311-314 is a predetermined distance from the location tool 120, each sensor 311-314 may detect a unique phase change to a sinusoidal acoustic wave that is being emitted by the location tool 120. The phase change is an indicator of how long the signal takes to reach the sensor 311-314 from the location tool 120. Since distance=velocity*time of waves, knowing the speed of sound through human tissue plus the time differential from the phase change may allow the three sensors 311-314 to accurately triangulate a geometric location within a two-dimensional plane. Additional (e.g., a fourth or fifth) sensors could be used for three-dimensional localization.

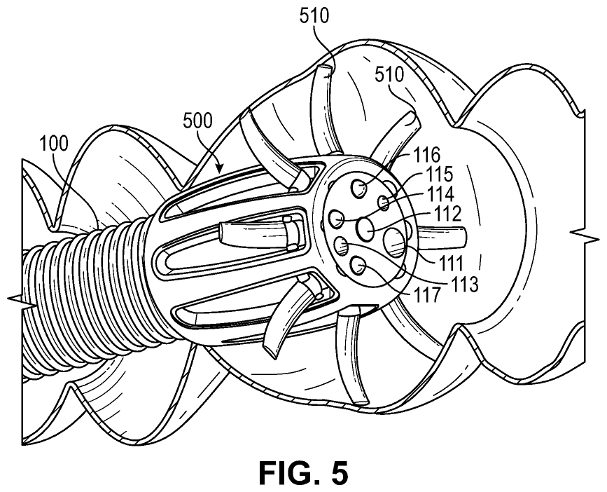

FIG. 4 illustrates a screen 400 showing data from the device 100 and the sensors 311-314. A first portion 410 of the screen 400 may show the images and/or video captured by the camera 130. In the example shown, the first portion 410 shows a polyp 414 inside the patient's colon 412. A second portion 420 of the screen 400 may show the location of the device 100 within the patient's abdomen 300. More particularly, the dashed line indicates the device 100 within the abdomen 300. The location tool 120 is positioned at a distal end of the device 100. Points 421-425 indicate the position of the location tool 120 at different times as the device 100 is pushed farther into the abdomen 300. The times may be, for example, 10 seconds apart.

FIG. 5 illustrates a perspective view of the device 100 from FIG. 1 with an endocuff 500 coupled to an end of the device 100. The bores 111-117 may also extend through the endocuff 500. The endocuff 500 may also include a plurality of fingers 510 that fan out as the endocuff 500 is being pulled out of the patient.

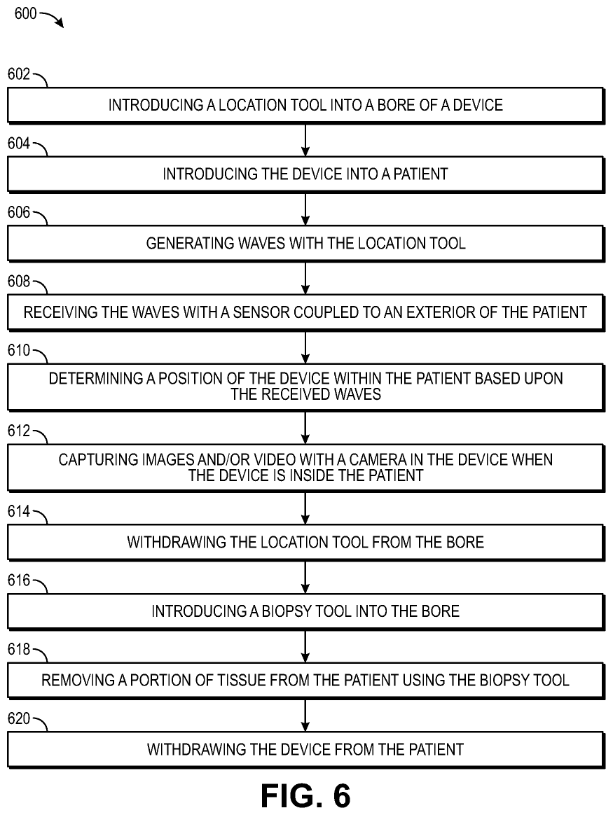

FIG. 6 illustrates a flowchart of a method 600 for operating the device 100 within a patient. The method 600 may include introducing the location tool 120 into the first bore 111 of the device 100, as at 602. The method 600 may then include introducing the device 100 into the patient through the patient's anus, as at 604. The method 600 may also include generating a plurality of waves with the location tool 120 when the device 100 is inside the patient, as at 606. As mentioned above, the waves may be generated by shining the laser 124 onto the cap 126. The method 600 may also include receiving the waves with the sensors 311-314 that are coupled to an exterior of the patient's abdomen 300, as at 608. The method 600 may also include determining the position of the device 100 within the patient based at least partially upon the waves received by the sensors 311-314, as at 610.

The method 600 may also include capturing images and/or video with the camera 130 when the device 100 is inside the patient, as at 612. When images and/or video reveal a portion of tissue (e.g., a polyp) that the doctor would like to analyze, the method 600 may include withdrawing the location tool 120 from the first bore 111 in the device 100 while the device 100 remains substantially stationary within the patient, as at 614. The method 600 may then include introducing a biopsy tool into the first bore 111 of the device 100 while the device 100 remains substantially stationary within the patient, as at 616. The method 600 may then include removing the portion of tissue from the patient using the biopsy tool, as at 618. The method 600 may then include withdrawing the device 100 from the patient, as at 620.

These steps can be carried out using a non-transitory computer readable medium loaded onto a computing device such as a personal computer, tablet, phablet, smartphone, computer server, or any other computing device known to or conceivable by one of skill in the art. Indeed, any suitable hardware and software known to or conceivable by one of skill in the art could be used. The non-transitory computer readable medium can also be incorporated into the device for assessment of PAT.

A non-transitory computer readable medium is understood to mean any article of manufacture that can be read by a computer. Such non-transitory computer readable media includes, but is not limited to, magnetic media, such as a floppy disk, flexible disk, hard disk, reel-to-reel tape, cartridge tape, cassette tape or cards, optical media such as CD-ROM, writable compact disc, magneto-optical media in disc, tape or card form, and paper media, such as punched cards and paper tape. The computing device can be a special computer designed specifically for this purpose. The computing device can be unique to the present invention and designed specifically to carry out the method of the present invention.

The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

What is claimed is:

1. A system for determining a location of a tool within a patient, comprising:
a tool configured to be introduced into a patient, wherein the tool comprises:
a flexible tubular member;
a laser coupled to the flexible tubular member; and
a cap coupled to the laser, wherein a plurality of waves are generated when a beam of light from the laser shines upon the cap; and
a first sensor configured to be coupled to an exterior of the patient, wherein the first sensor is configured to receive the waves, and wherein a location of the tool within the patient is configured to be determined based at least partially upon the waves received by the first sensor.

2. The system of claim 1, wherein the cap prevents the beam of light from the laser from shining on an interior of the patient.

3. The system of claim 1, further comprising a second sensor and a third sensor that are configured to be coupled to the exterior of the patient, wherein the first, second, and third sensors are configured to receive the waves, and wherein the location of the tool within the patient is configured to be determined by comparing phases of the waves received by the first, second, and third sensors.

4. The system of claim 3, wherein the first sensor is configured to be coupled to a sternum of the patient, the second sensor is configured to be coupled to a pubis of the patient, and the third sensor is configured to be coupled to an iliac crest of the patient.

5. The system of claim 1, wherein the tool is configured to generate the waves when the tool is positioned within a hollow organ in the patient.

6. The system of claim 1, wherein the tool is configured to generate the waves when the tool is positioned within a colon in the patient.

7. The system of claim 1, further comprising a flexible device that is configured to be introduced into the patient, wherein the device has a bore formed axially-therethrough, and wherein the tool is positioned within the bore.

8. The system of claim 7, wherein the tool is configured to be withdrawn from the bore while the device remains substantially stationary within the patient.

9. The system of claim 7, wherein the device comprises a colonoscope or an endocuff.

10. The system of claim 1, wherein the cap is metallic.

11. A method for operating a device within a patient, comprising:
introducing a device into a patient, wherein the device comprises a first bore having a location tool positioned therein;
generating a plurality of waves with the location tool when the device is positioned within the patient;
receiving the waves with a sensor coupled to an exterior of the patient; and
determining a position of the device within the patient based at least partially upon the waves received by the sensor.

12. The method of claim 11, wherein the device is introduced into the patient through the patient's anus.

13. The method of claim 12, wherein the device is positioned within a hollow organ in the patient.

14. The method of claim 11, wherein the location tool comprises a laser and a cap coupled to the laser, wherein the waves are generated when a beam of light from the laser shines upon the cap.

15. The method of claim 14, wherein the cap prevents the beam of light from the laser from shining on an interior of the patient.

16. The method of claim 14, further comprising capturing images, video, or both with a camera when the device is positioned within the patient, wherein the camera is positioned in a second bore in the device.

17. The method of claim 16, further comprising withdrawing the location tool from the first bore while the device remains substantially stationary within the patient.

18. The method of claim 17, further comprising:
introducing a biopsy tool into the first bore, after the location tool is withdrawn, while the device remains substantially stationary within the patient; and
removing a portion of tissue from the patient using the biopsy tool.

19. The method of claim 11, wherein the sensor comprises:
a first sensor coupled to a sternum of the patient;
a the second sensor coupled to a pubis of the patient; and
a third sensor coupled to an iliac crest of the patient.

20. The method of claim 19, wherein determining the position of the device comprises comparing phases of the waves received by the first, second, and third sensors to triangulate the position of the device.
